(19) United States
(12) Patent Application Publication
(10) Pub. No.: US 2024/0349962 A1
(43) Pub. Date: Oct. 24, 2024

(54) MUFFLER FOR AUTONOMOUS VACUUM

(71) Applicant: Matic Robots, Inc., Mountain View, CA (US)

(72) Inventors: Karthik Chandrashekaraiah, San Francisco, CA (US); Anshuman Kumar, San Francisco, CA (US); Zhen Bo Bian, Milpitas, CA (US); Mehul Nariyawala, Los Altos, CA (US); Navneet Dalal, Atherton, CA (US)

(21) Appl. No.: 18/641,811

(22) Filed: Apr. 22, 2024

Related U.S. Application Data
(60) Provisional application No. 63/497,680, filed on Apr. 21, 2023.

(51) Int. Cl. A47L 9/00

(52) U.S. Cl. CPC A47L 9/0081; A47L 2201/00

(57) ABSTRACT

A muffler for an autonomous vacuum includes one or more chambers and one or more noise-absorbing elements for noise reduction of air exhausted into an external environment of the autonomous vacuum. The muffler is coupled to a vacuum stack inclusive of a vacuum motor. The muffler includes a main body, a foam layer, and a sealing plate. The main body includes an inlet for air exhausted by the vacuum motor and forms the one or more chambers fluidically connected to the inlet. The foam layer is disposed within one of the chambers of the main body. The foam layer absorbs sound waves incident on the foam layer. The sealing plate is affixed to the main body and includes an outlet to exhaust air from the chambers to an external environment of the autonomous vacuum.

### CROSS REFERENCE TO RELATED APPLICATIONS

This present application claims the benefit of and priority to U.S. Provisional Application No. 63/497,680 filed on Apr. 21, 2023, which is incorporated by reference in its entirety.

### TECHNICAL FIELD

This disclosure relates to autonomous cleaning systems. More particularly, this disclosure relates to a mop roller implemented in an autonomous vacuum.

### BACKGROUND

Traditional autonomous cleaning systems typically are single functional, i.e., focused separately on vacuuming or on mopping. Systems that attempt to combine the two functionalities, nonetheless, decouple components directed at each functionality, such that one subset of components focuses on the vacuuming, while another subset focuses on the mopping. The former systems might be efficient in their specific mode, but are incapable of tackling messes of the other type. The latter systems typically sacrifice one cleaning function to promote the other. Either way, these systems are typically clunky incorporating two distinct sets of components. There remains a need for an efficient combinative system.

In particular, there remains a need for improved noise management solutions to minimize the noise created from operation of the vacuum motor. The noise from the turbulent airflow generated by vacuum motor is one of the primary sources of noise generation in the cleaning robot. For an efficient cleaning system, high airflow is needed. The inevitable downside is that high airflow causes higher turbulence and higher noise.

### BRIEF DESCRIPTION OF THE DRAWINGS

The disclosed embodiments have other advantages and features which will be more readily apparent from the detailed description, the appended claims, and the accompanying figures (or drawings). A brief introduction of the figures is below.

FIG. 1 is a block diagram of an autonomous vacuum, according to one or more embodiments.

FIG. 2 illustrates a spatial arrangement of components of the autonomous vacuum, according to one or more embodiments.

FIG. 3 is a block diagram of a sensor system of the autonomous vacuum, according to one or more embodiments.

FIG. 4 is a block diagram of a controller of the autonomous vacuum, according to one or more embodiments.

FIG. 5 is a cross-sectional view of an airflow pathway for the autonomous vacuum, according to one or more embodiments.

FIG. 6A is an exploded view of a muffler for dampening noise created by the vacuum motor, according to one or more embodiments.

FIG. 6B is a constructed view of the muffler, according to one or more embodiments.

FIG. 7A is a cross-sectional view of the muffler along a first axis, according to one or more embodiments.

FIG. 7B is a cross-sectional view of the muffler along a second axis perpendicular to the first axis, according to one or more embodiments.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### CONFIGURATION OVERVIEW

An autonomous vacuum capable of both vacuuming and mopping implements a dual-roller cleaning head, one for dry cleaning and another for wet cleaning, both configured to ingest waste into one waste bag. The autonomous vacuum utilizes vacuum force to ingest both dry waste and liquid waste from the cleaning head into the waste bag. To maintain high cleaning efficiency, the vacuum motor is operated at a high speed. To counteract noise generated by the high-speed operation, the autonomous vacuum includes a muffler designed for maximal noise reduction.

The muffler includes one or more chambers and one or more noise-absorbing elements for noise reduction of air exhausted into an external environment of the autonomous vacuum. The muffler is coupled to a vacuum stack inclusive of a vacuum motor. The muffler includes a main body, a foam layer, and a sealing plate. The main body includes an inlet for air exhausted by the vacuum motor and forms the one or more chambers fluidically connected to the inlet. The foam layer is disposed within one of the chambers of the main body. The foam layer absorbs sound waves incident on the foam layer. The sealing plate is affixed to the main body and includes an outlet to exhaust air from the chambers to an external environment of the autonomous vacuum. The muffler may include other noise-absorbing elements, e.g., a fabric layer coupled to a rigid layer separating two chambers in the muffler, or a foam installed in the outlet of the muffler, etc.

### AUTONOMOUS VACUUM ARCHITECTURE

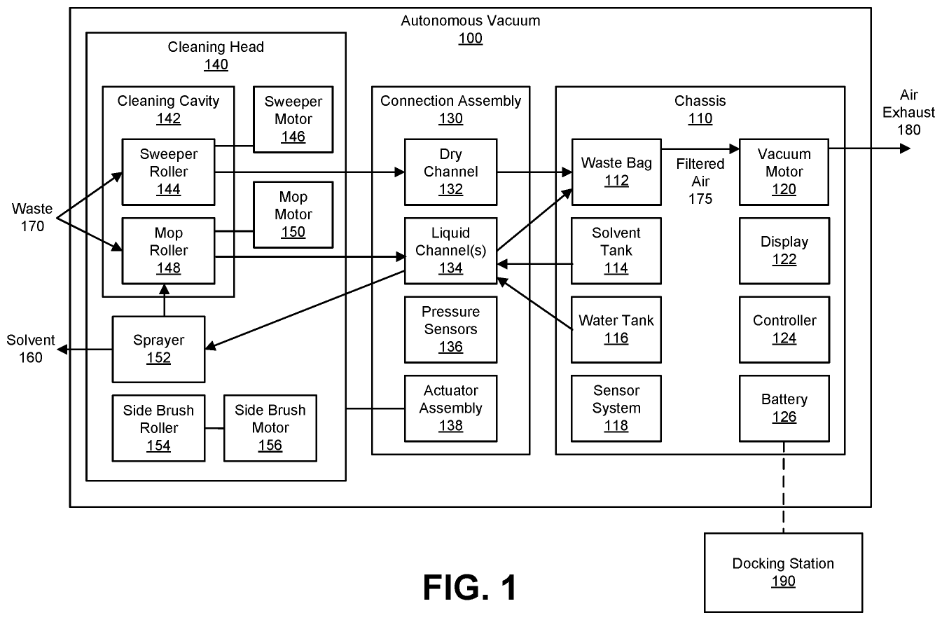

FIG. 1 is a block diagram of an autonomous vacuum 100, according to one or more embodiments. The autonomous vacuum 100 in this example may include a chassis 110, a connection assembly 130, and a cleaning head 140. The components of the autonomous vacuum 100 allow the autonomous vacuum 100 to intelligently clean as the autonomous vacuum 100 traverses an area within an environment.

As an overview, the chassis 110 is a rigid body that serves as a base frame for the autonomous vacuum. The chassis 110 comprises a plurality of motorized wheels for driving the autonomous vacuum 100. The chassis 110 hosts a suite of other components for navigating the autonomous vacuum 100, communicating with external devices, providing notifications, among other operations. The connection assembly 130 serves as a connection point between the cleaning head 140 and the chassis 110. The connection assembly 130 comprises at least a plurality of channels used to direct solvent, water, waste, or some combination thereof between the cleaning head 140 and the chassis 110. The connection assembly 130 also comprises an actuator assembly 138 that controls movement of the cleaning head 140. The cleaning head 140 comprises the one or more brush rollers used to perform cleaning operations. In some embodiments, the architecture of the autonomous vacuum 100 includes more components for autonomous cleaning purposes. Some examples include a mop roller, a solvent dispersion system, a waste container, a muffler, and multiple solvent containers for different types of cleaning solvents. It is noted that the autonomous vacuum 100 may include functions that include cleaning functions that include, for example, vacuuming, sweeping, dusting, mopping, and/or deep cleaning.

The chassis 110 is a rigid body as a base frame for the autonomous vacuum 100. In one or more embodiments, the chassis 110 comprises at least a waste bag 112, a solvent tank 114, a water tank 116, a sensor system 118, a vacuum motor 120, a display 122, a controller 124, and a battery 126. In other embodiments, the chassis 110 may comprise additional, fewer, or different components than those listed herein. For example, one embodiment of the chassis 110 omits the display 122. Or another embodiment includes an additional output device, such as a speaker. Still another embodiment may combine the solvent tank 114 and the water tank 116 into a single tank. In yet another embodiment, the chassis 110 may include a muffler for the vacuum motor 120, to passively dampen noise created by operating the vacuum motor 120.

The waste bag 112 collects the waste that is accumulated from performing cleaning routines. The waste bag 112 may be configured to collect solid and/or liquid waste. In one or more embodiments, there may be two separate waste bags, one for solid waste and one for liquid waste. The waste bag 112 may be removably secured within the chassis 110. As the waste bag 112 is filled, the autonomous vacuum 100 may alert the user to empty the waste bag 112 and/or replace the waste bag 112. In other embodiments, the waste bag 112 can remain in the chassis 110 when emptied. In such embodiments, the chassis 110 may further comprise a drainage channel connected to the waste bag to drain the collected waste. The waste bag 112 may further comprise an absorbent material that soaks up liquid, e.g., to prevent the liquid from sloshing out of the bag during operation of the autonomous vacuum 100.

The solvent tank 114 comprises solvent used for cleaning. The solvent tank 114 comprises at least a chamber and one or more valves for dispensing from the chamber. The solvent is a chemical formulation used for cleaning. Example solvents include dish detergent, soap, bleach, other organic and/or nonorganic solvents. In some embodiments, the solvent tank 114 comprises dry solvent that is mixed with water from the water tank 116 to create a cleaning solution. The solvent tank may be removable, allowing a user to refill the solvent tank 114 when the solvent tank 114 is empty.

The water tank 116 stores water used for cleaning. The water tank 116 comprises at least a chamber and one or more valves for dispensing from the chamber. The water tank 116 may be removable, allowing a user to refill the water tank 116 when the water tank 116 is empty. In one or more embodiments, the water tank 116 comprises a valve located on the bottom of the water tank 116, when the water tank 116 is secured in the chassis 110. The weight of the water applies a downward force due to gravity, a spring mechanism, or some combination thereof, which keeps the valve closed. To open the valve, some protrusion on the chassis 110 applies a counteracting upward force that opens the valve, e.g., by pushing the valve towards an interior of the chamber revealing an outlet permitting water to escape the water tank 116.

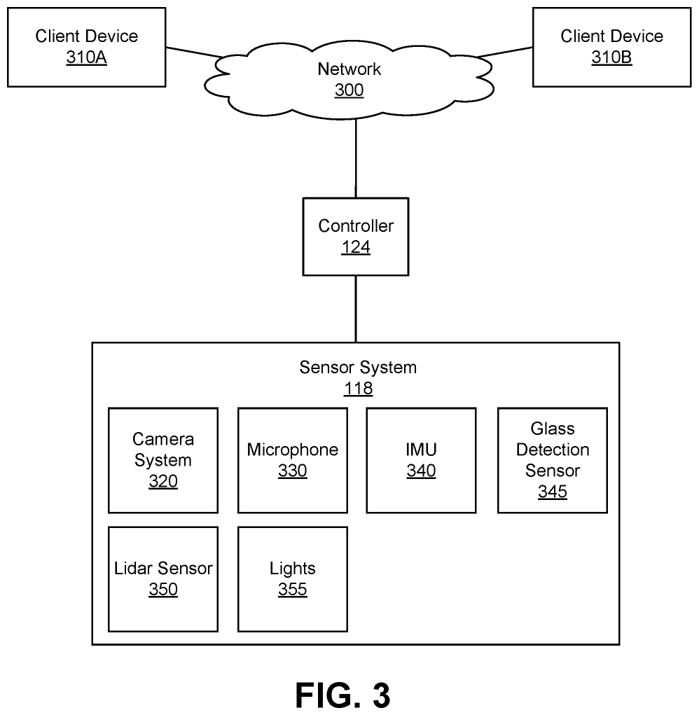

The sensor system 118 comprises a suite of sensors for guiding operation of the autonomous vacuum 100. Each sensor captures sensor data that can be fed to the controller 124 to guide operation of the autonomous vacuum 100. The sensor system 118 is further described in FIG. 3.

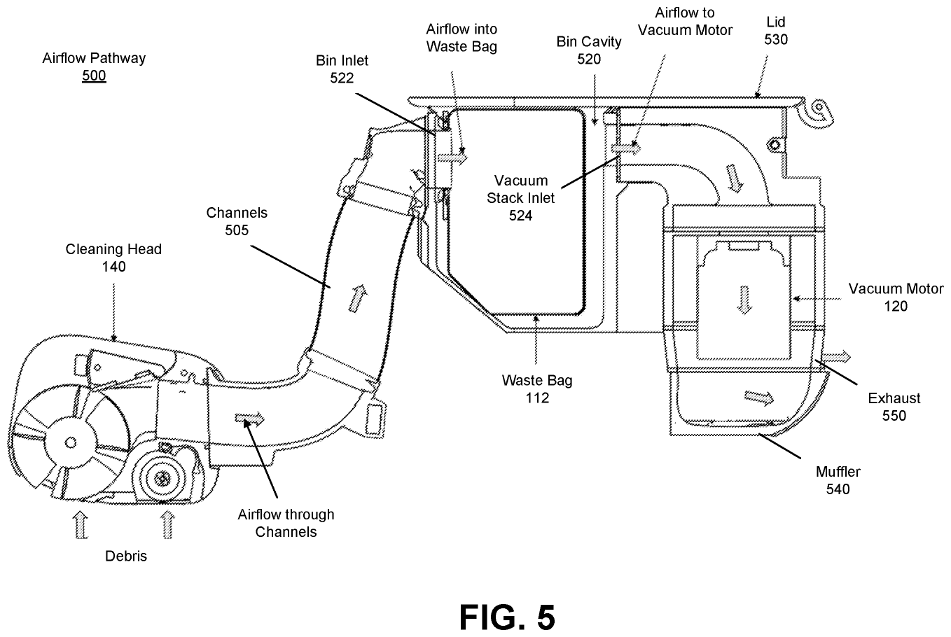

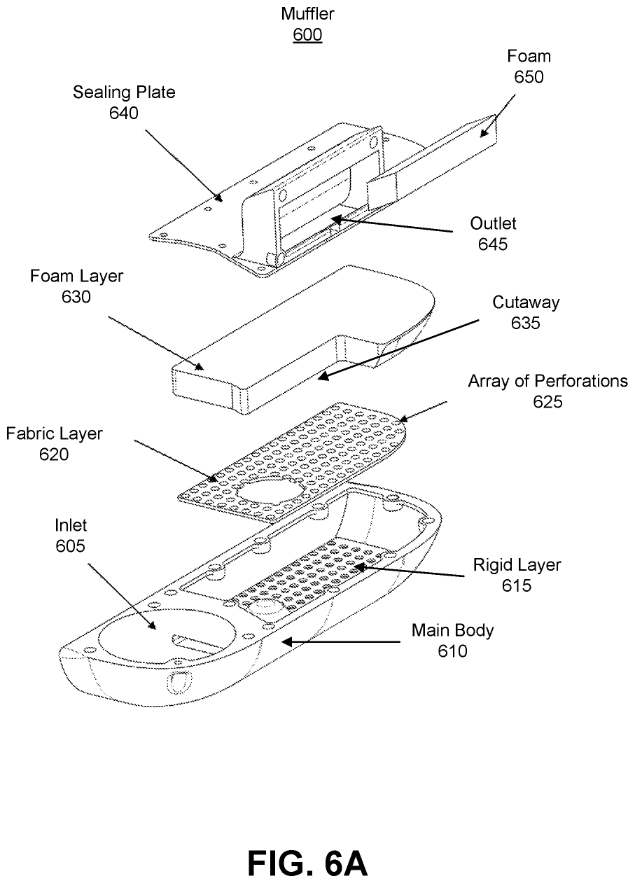

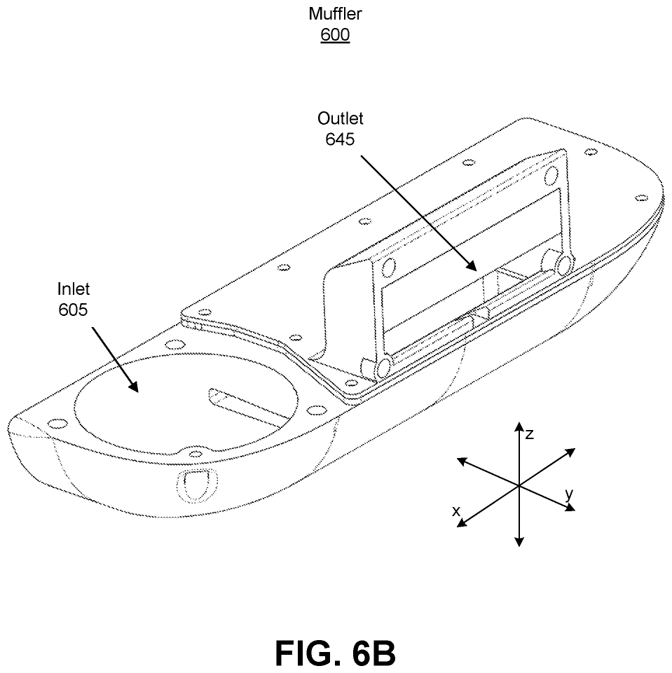

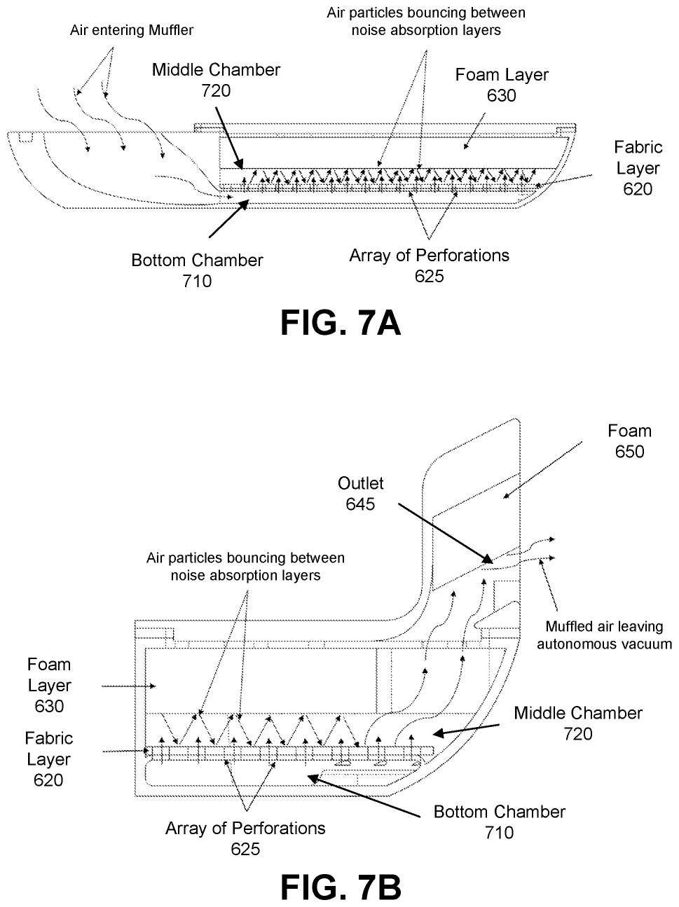

The vacuum motor 120 generates a vacuum force that aids ingestion of waste by the cleaning head 140. In one or more embodiments, to generate the vacuum force, the vacuum motor 120 may comprise one or more fans that rotate to rapidly move air. The vacuum force flows through the waste bag 112, through the connection assembly 130, and to the cleaning head 140. An example airflow pathway is illustrated in FIG. 5. Furthermore, the vacuum motor 120 may include a muffler coupled to a backend of the vacuum motor 120 (in the airflow pathway) that passively dampens noise created by the vacuum motor 120, prior to exhausting air out of the autonomous vacuum 100. Detailed embodiments of the muffler are described in conjunction with FIGS. 6A, 6B, 7A, and 7B.

The display 122 is an electronic display that can display visual content. The display 122 may be positioned on a topside of the autonomous vacuum 100. The display may be configured to notify a user regarding operation of the autonomous vacuum 100. For example, notifications may describe an operation being performed by the autonomous vacuum 100, an error message, the health of the autonomous vacuum 100, etc. The display 122 may be an output device that includes a driver and/or screen to drive presentation of (e.g., provides for display) and/or present visual information. The display 122 may include an application programming interface (API) that allows users to interact with and control the autonomous vacuum. In some embodiments, the display may additionally or alternatively include physical interface buttons along with a touch sensitive interface. The display 122 receives data from the sensor system 118 and may display the data via the API. The data may include renderings of a view (actual image or virtual) of a physical environment, a route of the autonomous vacuum 100 in the environment, obstacles in the environment, and messes encountered in the environment. The data may also include alerts, analytics, and statistics about cleaning performance of the autonomous vacuum 100 and messes and obstacles detected in the environment.

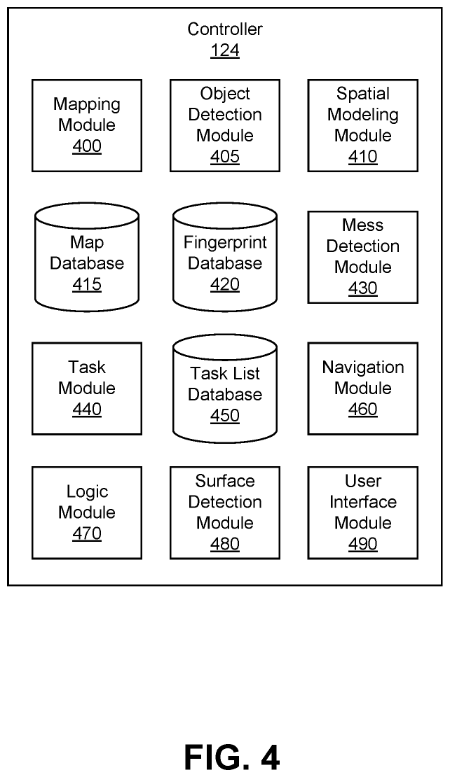

The controller 124 is a general computing device that controls operation of the autonomous vacuum 100. As a general computing device, the controller 124 may comprise at least one or more processors and computer-readable storage media for storing instructions executable by the processors. Operations of the controller 124 include navigating the autonomous vacuum 100, simultaneous localization and mapping of the autonomous vacuum 100, controlling operation of the cleaning head 140, generating notifications to provide to the user via one or more output devices (e.g., the display 122, a speaker, or a notification transmittable to the user's client device, etc.), running quality checks on the various components of the autonomous vacuum 100, controlling docking at the docking station 190, etc. Details of the controller 124 are described in FIG. 4.

The controller 124 may control movement of the autonomous vacuum 100. In particular, the controller connects to one or more motors connected to one or more wheels that may be used to move the autonomous vacuum 100 based on sensor data captured by the sensor system 118 (e.g., indicating a location of a mess to travel to). The controller may cause the motors to rotate the wheels forward/backward or turn to move the autonomous vacuum 100 in the environment. Based on surface type detection by the sensor system 118, the controller 124 may modify or alter navigation of the autonomous vacuum 100.

The controller of the actuator assembly 138 may also control cleaning operations. Cleaning operations may include a combination of rotation of the brush rollers, positioning or orienting the cleaning head 140 via the actuator assembly 138, controlling dispersion of solvent, activation of the vacuum motor 120, monitoring the sensor system 118, other functions of the autonomous vacuum, etc.

In controlling rotation of the brush rollers, the controller 124 may connect to one or more motors (e.g., the sweeper motor 146, the mop motor 150, and the side brush motor 156) positioned at the ends of the brush rollers. The controller 124 can toggle rotation of the brush rollers between rotating forward or backward or not rotating using the motors. In some embodiments, the brush rollers may be connected to an enclosure of the cleaning head 140 via rotation assemblies each comprising one or more of direct drive, geared or belted drive assemblies that connect to the motors to control rotation of the brush rollers. The controller 124 may rotate the brush rollers based on a direction needed to clean a mess or move a component of the autonomous vacuum 100. In some embodiments, the sensor system 118 determines an amount of pressure needed to clean a mess (e.g., more pressure for a stain than for a spill), and the controller 124 may alter the rotation of the brush rollers to match the determined pressure. The controller 124 may, in some instances, be coupled to a load cell at each brush roller used to detect pressure being applied by the brush roller. In another instance, the sensor system 118 may be able to determine an amount of current required to spin each brush roller at a set number of rotations per minute (RPM), which may be used to determine a pressure being exerted by the brush roller. The sensor system 118 may also determine whether the autonomous vacuum 100 is able to meet an expected movement (e.g., if a brush roller is jammed) and adjust the rotation via the controller if not. Thus, the sensor system 118 may optimize a load being applied by each brush roller in a feedback control loop to improve cleaning efficacy and mobility in the environment. The controller 124 may additionally control dispersion of solvent during the cleaning operation by controlling a combination of the sprayer 152, the liquid channels 134, the solvent tank 114, the water tank 116, and turning on/off the vacuum motor 120.

The battery 126 stores electric charge for powering the autonomous vacuum 100. The battery 126 may be rechargeable when the autonomous vacuum 100 is docked at the docking station 190. The battery 126 may implement a battery optimization scheme to efficiently distribute power across the various components. The autonomous vacuum 100 is powered with an internal battery 126. The battery 126 stores and supplies electrical power for the autonomous vacuum 100. In some embodiments, the battery 126 consists of multiple smaller batteries that charge specific components of the autonomous vacuum 100.

The autonomous vacuum 100 may dock at a docking station 190 to charge the battery 126. The docking station 190 may be connected to an external power source to provide power to the battery 126. External power sources may include a household power source and one or more solar panels. The docking station 190 also may include processing, memory, and communication computing components that may be used to communicate with the autonomous vacuum 100 and/or a cloud computing infrastructure (e.g., via wired or wireless communication). These computing components may be used for firmware updates and/or communicating maintenance status.

The docking station 190 also may include other components, such as a cleaning station for the autonomous vacuum 100. In some embodiments, the cleaning station includes a solvent tray that the autonomous vacuum 100 may spray solvent into and roll the roller 144 or the side brush roller 154 in for cleaning. In other embodiments, the autonomous vacuum may eject the waste bag 112 into a container located at the docking station 190 for a user to remove.

The connection assembly 130 is a rigid body that connects the cleaning head 140 to the chassis 110. A four-bar linkage may join the cleaning head 140 to the connection assembly 130. In one or more embodiments, the connection assembly 130 comprises at least a dry channel 132, one or more liquid channels 134, one or more pressure sensors 136, and an actuator assembly. Channel refers generally to either a dry channel or a liquid channel. In other embodiments, the connection assembly 130 may comprise additional, fewer, or different components than those listed herein. For example, one or more sensors of the sensor system 118 may be disposed on the connection assembly 130.

The dry channel 132 is a conduit for dry waste from the cleaning head 140 to the waste bag 112. The dry channel 132 is substantially large in diameter to permit movement of most household waste.

The one or more liquid channels 134 are conduits for liquids between the cleaning head 140 and the chassis 110. There is at least one liquid channel 134 (a liquid waste channel) that carries liquid waste from the cleaning head 140 to the waste bag 112. In one or more embodiments, the liquid channel 134 carrying liquid waste may be smaller in diameter than the dry channel 132. In such embodiments, the autonomous vacuum 100 sweeps (collecting dry waste) before mopping (collecting liquid waste). There is at least one other liquid channel 134 (a liquid solution channel) that carries water, solvent, and/or cleaning solution (combination of water and solvent) from the chassis 110 to the cleaning head 140 for dispersal to the cleaning environment.

The one or more pressure sensors 136 measure pressure in one or more of the channels. The pressure sensors 136 may be located at various positions along the connection assembly 130. The pressure sensors 136 provide the pressure measurements to the controller 124 for processing.

The actuator assembly 138 controls movement and position of the cleaning head 140, relative to the chassis 110. The actuator assembly 138 comprises at least one or more actuators configured to generate linear and/or rotational movement of the cleaning head 140. Linear movement may include vertical height of the cleaning head 140. Rotational movement may include pitching the cleaning head 140 to varying angles, e.g., to switch between sweeping mope and mopping mode, or to adjust cleaning by the cleaning head 140 based on detected feedback signals. The actuator assembly 138 may comprise a series of joints that aid in providing the movement to the cleaning head 140.

The cleaning head 140 performs one or more cleaning operations to clean an environment. The cleaning head 140 is also a rigid body that forms a cleaning cavity 142, where a sweeper roller 144 and a mop roller 148 are disposed. The cleaning head 140 further comprises a sweeper motor 146, a mop motor 150, a sprayer 152, a side brush roller 154, and a side brush motor 156. Collectively, the sweeper roller 144, the mop roller 148, and the side brush roller 154 are referred to as the brush rollers. Likewise, the brush motors include the sweeper motor 146, the mop motor 150, and the side brush motor 156. In some embodiments, each brush roller may be composed of different materials and operate at different times and/or speeds, depending on a cleaning task being executed by the autonomous vacuum 100. In other embodiments, the cleaning head 140 comprises additional, fewer, or different components than those listed herein. In some embodiments, the autonomous vacuum 100 may include two or more sweeper rollers 144 controlled by two or more sweeper motors 146. In some embodiments, the cleaning head 140 may be referred to as a roller housing.

The sweeper roller 144 sweeps dry waste into the autonomous vacuum 100. The sweeper roller 144 generally comprises one or more brushes attached to a cylindrical core. The sweeper roller 144 rotates to collect and clean messes. The sweeper roller 144 may be used to handle large particle messes, such as food spills or small plastic items like bottle caps. When the sweeper roller 144 is activated by the sweeper motor 146, the brushes act in concert to sweep dry waste towards a dry inlet connected to the dry channel 132. The brushes may be composed of a compliant material to sweep the dry waste. In some embodiments, the sweeper roller 144 may be composed of multiple materials for collecting a variety of waste, including synthetic bristle material, microfiber, wool, or felt.

The mop roller 148 mops the cleaning environment and ingests liquid waste into the autonomous vacuum 100. The mop roller 148 generally comprises fabric bristles attached to a cylindrical core. With the aid of a cleaning solution, the fabric bristles work to scrub away dirt, grease, or other contaminants that may have stuck to the cleaning surface. The mop motor 150 provides rotational force to the mop roller 148. In some embodiments, the mop roller 148 may be composed of multiple materials for collecting a variety of waste, including synthetic bristle material, microfiber, wool, or felt.

In normal sweeping mode, as the air flows from the dry channel 132 and the dry inlet towards the vacuum motor 120, the sweeper roller 144 rotates to move dry waste from the cleaning environment towards the inlet, in order to deposit the dry waste in the waste bag 112. In normal mopping mode, the cleaning head 140 sprays the cleaning solution (solvent or solvent mixed with water) onto the cleaning environment or on top of the mop roller itself. The mop roller 148 contacts the sprayed surface to scrub the surface with the fabric bristles. The vacuum force sucks up or ingests the liquid waste to deposit the liquid waste into the waste bag 112.

The side brush roller 154 sweeps dirt near a side of the cleaning head 140. The side brush roller 154 may rotate along an axis that is orthogonal or perpendicular to the ground. The side brush that is controlled by a side brush motor 156. The side brush roller 154 may be shaped like a disk or a radial arrangement of bristles that can push dirt into the path of the sweeper roller 144. In some embodiments, the side brush roller 154 is composed of different materials than the sweeper roller 144 to handle different types of waste and mess. In one or more embodiments, the side brush roller 154 may be concealed to minimize a profile of the cleaning head 140 when the side brush roller 154 is not in operation.

The sprayer 152 sprays liquid into the cleaning environment. The sprayer 152 is connected to the liquid solution channel that is connected to the solvent tank 114 and/or the water tank 116. A pump on the chassis 110 can dispense solvent and/or water from the solvent tank 114 and/or the water tank 116. The liquid travels to the sprayer 152, which then has a nozzle for spraying the liquid into the cleaning environment. The sprayer 152 may include a plurality of nozzles, e.g., two disposed on either side of the cleaning head.

The cleaning head 140 ingests waste 155 as the autonomous vacuum 100 cleans using the roller 144 and the side brush roller 154 and sends the waste 155 to the waste bag 112. The waste bag 112 collects and filters waste 155 from the air to send filtered air 165 out of the autonomous vacuum 100 through the vacuum motor 120 as air exhaust 170. The autonomous vacuum 100 may also use solvent 160 combined with pressure from the cleaning head 140 to clean a variety of surface types. The autonomous vacuum may dispense solvent 160 from the solvent tank 114 onto an area to remove dirt, such as dust, stains, and solid waste and/or clean up liquid waste. The autonomous vacuum 100 may also dispense solvent 160 into a separate solvent tray, which may be part of a charging station (e.g., docking station 190), described below, clean the roller 144 and the side brush roller 154.

Mess types are the form of mess in the environment, such as smudges, stains, and spills. It also includes the type of phase the mess embodies, such as liquid, solid, semi-solid, or a combination of liquid and solid. Some examples of waste include bits of paper, popcorn, leaves, and particulate dust. A mess typically has a size/form factor that is relatively small compared to obstacles that are larger. For example, spilled dry cereal may be a mess but the bowl it came in would be an obstacle. Spilled liquid may be a mess, but the glass that held it may be an obstacle. However, if the glass broke into smaller pieces, the glass would then be a mess rather than an obstacle. Further, if the sensor system 118 determines that the autonomous vacuum 100 cannot properly clean up the glass, the glass may again be considered an obstacle, and the sensor system 118 may send a notification to a user indicating that there is a mess that needs user cleaning. The mess may be visually defined in some embodiments, e.g., visual characteristics. In other embodiments it may be defined by particle size or make up. When defined by size, in some embodiments, a mess and an obstacle may coincide. For example, a small interlocking brick piece may be the size of both a mess and an obstacle. The sensor system 118 is further described in relation to FIG. 3.

The actuator assembly 138 includes one or more actuators (henceforth referred to as an actuator for simplicity), one or more controllers and/or processors (henceforth referred to as a controller for simplicity) that operate in conjunction with the sensor system 118 to control movement of the cleaning head 140. In particular, the sensor system 118 collects and uses sensor data to determine an optimal height for the cleaning head 140 given a surface type, surface height, and mess type. Surface types may be the floorings used in the environment and may include surfaces of varying characteristics (e.g., texture, material, absorbency), for example, carpet, wood, tile, rug, laminate, marble, and vinyl.

The actuator assembly 138 automatically adjusts the height of the cleaning head 140 given the surface type, surface height, and mess type. In particular, the actuator controls vertical movement and rotation tilt of the cleaning head 140. The actuator may vertically actuate the cleaning head 140 based on instructions from the sensor system 118. For example, the actuator may adjust the cleaning head 140 to a higher height if the sensor system 118 detects thick carpet in the environment than if the processor detects thin carpet. Further, the actuator may adjust the cleaning head 140 to a higher height for a solid waste spill than a liquid waste spill. In some embodiments, the actuator may set the height of the cleaning head 140 to push larger messes out of the path of the autonomous vacuum 100. For example, if the autonomous vacuum 100 is blocked by a pile of books, the sensor system 165 may detect the obstruction (i.e., the pile of books) and the actuator may move the cleanings head 105 to the height of the lowest book, and the autonomous vacuum 100 may move the books out of the way to continue cleaning an area. Furthermore, the autonomous vacuum 100 may detect the height of obstructions and/or obstacles, and if an obstruction or obstacle is over a threshold size, the autonomous vacuum 100 may use the collected visual data to determine whether to climb or circumvent the obstruction or obstacle by adjusting the cleaning head height using the actuator assembly 138.

In other embodiments, any of the components of the autonomous vacuum can be variable distributed among the chassis 110, the connection assembly 130, and the cleaning head 140.

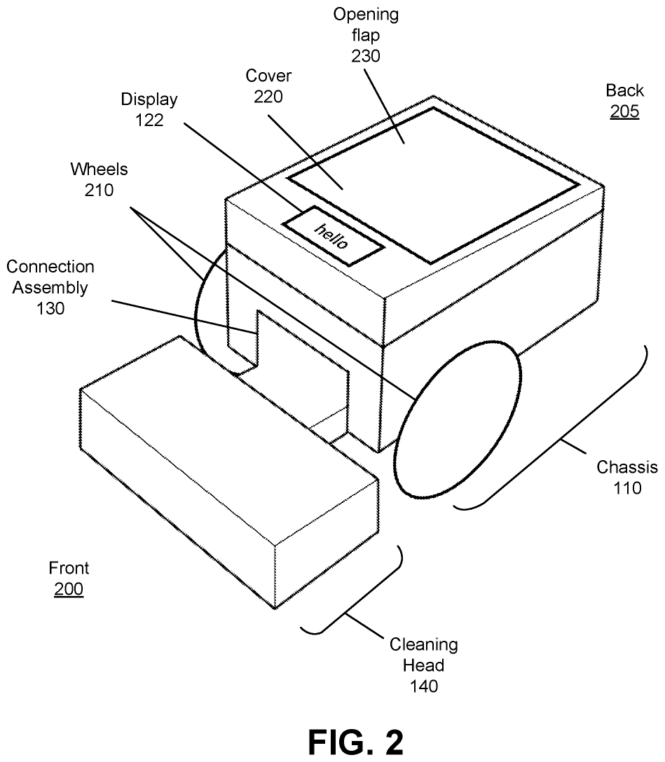

FIG. 2 illustrates a spatial arrangement of components of the autonomous vacuum 100, according to one or more embodiments. The autonomous vacuum 100 includes the cleaning head 140 (as described in relation to FIG. 1) at the front 200 and the chassis 110 at the back 205. The cleaning head 140 may be connected to the chassis 110 via the connection assembly 130 (e.g., a four-bar linkage system). The connection assembly 130 may be connected to one or more actuators of the actuator assembly 138 such that the actuators can control movement of the cleaning head 140 with the four-bar linkage system. Movement includes vertical translation of the cleaning head 140, i.e., towards or away from a ground plane, and rotation of the cleaning head 140, e.g., about a rotational axis established by a pivot point with the four-bar linkage system.

The chassis 110 includes the frame, a plurality of wheels 210, a cover 220, and opening flap 230, and a display 122. In particular, the cover 220 is an enclosed hollow structure that covers containers internal to the base that contain solvent and waste (e.g., in the waste bag 112). The opening flap 230 that may be opened or closed by a user such that the user can access the containers (e.g., to add more solvent, remove the waste bag 112, or put in a new waste bag 112). The cover may also house a subset of the sensors of the sensor system 118 and the actuator assembly 138, which may be configured at a front of the cover 220 to connect to the cleaning head 140. The display 122 is embedded in the cover 220 of the autonomous vacuum 100 and may include physical interface buttons and a touch sensitive interface.

### Sensor System

FIG. 3 is a block diagram of the sensor system 118 of the autonomous vacuum 100, according to one or more embodiments. The sensor system 118 receives sensor data from, for example, one or more cameras (video/visual), microphones 330 (audio), lidar sensors infrared (IR) sensors, and/or inertial sensors that capture inertial data (e.g., environmental surrounding or environment sensor data) about an environment for cleaning. The sensor system 118 uses the sensor data to map the environment and determine and execute cleaning tasks to handle a variety of messes. The controller 124 manages operations of the sensor system 118 and its various components. The controller 124 may communicate with one or more client devices 310 via a network 300 to send sensor data, alert a user to messes, or receive cleaning tasks to add to a task list.

The network 300 may comprise any combination of local area and/or wide area networks, using wired and/or wireless communication systems. In one embodiment, the network 300 uses standard communications technologies and/or protocols. For example, the network 300 includes communication links using technologies such as Ethernet, 802.11 (WiFi), worldwide interoperability for microwave access (WiMAX), 3G, 4G, 5G, code division multiple access (CDMA), digital subscriber line (DSL), Bluetooth, Near Field Communication (NFC), Universal Serial Bus (USB), or any combination of protocols. In some embodiments, all or some of the communication links of the network 300 may be encrypted using any suitable technique or techniques.

The client device 310 is a computing device capable of receiving user input as well as transmitting and/or receiving data via the network 300. Though only two client devices 310 are shown in FIG. 4, in some embodiments, more or less client devices 310 may be connected to the autonomous vacuum 100. In one embodiment, a client device 310 is a conventional computer system, such as a desktop or a laptop computer. Alternatively, a client device 310 may be a device having computer functionality, such as a personal digital assistant (PDA), a mobile telephone, a smartphone, a tablet, an Internet of Things (IoT) device, or another suitable device. A client device 310 is configured to communicate via the network 300. In one embodiment, a client device 310 executes an application allowing a user of the client device 310 to interact with the sensor system 118 to view sensor data, receive alerts, set cleaning settings, and add cleaning tasks to a task list for the autonomous vacuum 100 to complete, among other interactions. For example, a client device 310 executes a browser application with an application programming interface (API) that enables interactions between the client device 310 and the autonomous vacuum 100 via the network 300. In another embodiment, a client device 310 interacts with autonomous vacuum 100 through an application running on a native operating system of the client device 310, such as iOS® or ANDROID™.

In one or more embodiments, the sensor system 118 includes a camera system 320, microphone 330, inertial measurement device (IMU) 340, a glass detection sensor 345, a lidar sensor 350, and lights 355. The sensor system 118 may further include other sensors of the autonomous vacuum 100, e.g., the pressure sensors disposed in the connection assembly 130.

The camera system 320 comprises one or more cameras that capture images and or video signals as visual data about the environment. In some embodiments, the camera system 320 includes an IMU (separate from the IMU 340 of the sensor system 118) for capturing visual-inertial data in conjunction with the cameras. The visual data captured by the camera system 320 may be used by storage medium for image processing, as described in relation to FIG. 4.

The microphone 330 captures audio data by converting sound into electrical signals that can be stored or processed by other components of the sensor system 118. The audio data may be processed to identify voice commands for controlling functions of the autonomous vacuum 100, as described in relation to FIG. 4. In an embodiment, sensor system 118 uses more than one microphone 330, such as an array of microphones.

The IMU 340 captures inertial data describing the autonomous vacuum's 100 force, angular rate, and orientation. The IMU 340 may comprise of one or more accelerometers, gyroscopes, and/or magnetometers. In some embodiments, the sensor system 118 employs multiple IMUs 340 to capture a range of inertial data that can be combined to determine a more precise measurement of the autonomous vacuum's 100 position in the environment based on the inertial data.

The glass detection sensor 345 detects glass in the environment. Glass may be transparent material that may be stained, leaded, laminate or the like and may be part of furniture, flooring, or other objects in the environment (e.g., cups, mirrors, candlesticks, etc.). The glass detection sensor 345 may be an infrared sensor and/or an ultrasound sensor. In some embodiments, the glass detection sensor 345 is coupled with the camera system 320 to remove glare from the visual data when glass is detected. For example, the camera system 320 may have integrated polarizing filters that can be applied to the cameras of the camera system 320 to remove glare. In some embodiments, the glass sensor is a combination of an IR sensor and neural network that determines if an obstacle in the environment is transparent (e.g., glass) or opaque.

The lidar sensor 350 emits pulsed light into the environment and detects reflections of the pulsed light on objects (e.g., obstacles or obstructions) in the environment. Lidar data captured by the lidar sensor 350 may be used to determine a 3D representation of the environment. The lights 355 are one or more illumination sources that may be used by the autonomous vacuum 100 to illuminate an area around the autonomous vacuum 100. In some embodiments, the lights may be LEDs, e.g., having a static color such as white or green, or changeable colors (such as green of operating, red for stopped and yellow indicating slowing down).

The controller 124 may control the sensor system 118 various functions attributed to the sensor system 118 described herein. For example, a storage medium may store one or more modules or applications (described in relation to FIG. 4) embodied as instructions executable by a processor. The instructions, when executed by the processor, cause the processor to carry out the functions attributed to the various modules or applications described herein or instruct the controller and/or actuator to carry out movements and/or functions. For example, instructions may include when to take the sensor data, where to move the autonomous vacuum 100 to, and how to clean up a mess. In one embodiment, the processor may comprise a single processor or a multi-processor system.

### CONTROLLER ARCHITECTURE

FIG. 4 is a block diagram of the controller 124, according to one or more embodiments. The controller 124 includes a mapping module 400, an object detection module 405, a spatial modeling module 410, a map database 415, a fingerprint database 420, a mess detection module 430, a task module 440, a task list database 450, a navigation module 460, a logic module 470, a surface detection module 480, and a user interface module 490. In some embodiments, the controller 124 includes other modules that control various functions for the autonomous vacuum 100. Examples include a separate image processing module, a separate command detection module, and an object database.

The mapping module 400 creates and updates a map of an environment as the autonomous vacuum 100 moves around the environment. The map may be a two-dimensional (2D) or a three-dimensional (3D) representation of the environment including objects and other defining features in the environment. For simplicity, the environment may be described in relation to a house in this description, but the autonomous vacuum 100 may be used in other environments in other embodiments. Example environments include offices, retail spaces, and classrooms. For a first mapping of the environment, the mapping module 400 receives visual data from the camera system 320 and uses the visual data to construct a map. In some embodiments, the mapping module 400 also uses inertial data from the IMU 340 and lidar and IR data to construct the map. For example, the mapping module 400 may use the inertial data to determine the position of the autonomous vacuum 100 in the environment, incrementally integrate the position of the autonomous vacuum 100, and construct the map based on the position. However, for simplicity, the data received by the mapping module 400 will be referred to as visual data throughout the description of this figure.

In another embodiment, the mapping module 400 may capture a 360 degree “panorama view” using the camera system 320 while the autonomous vacuum 100 rotates around a center axis. The mapping module 400 applies a neural network to the panorama view to determine a boundary within the environment (e.g., walls), which the mapping module 400 may use for the representation of the environment. In other embodiments, the mapping module 400 may cause the autonomous vacuum 100 to trace the boundary of the environment by moving close to walls or other bounding portions of the environment using the camera system 100. The mapping module 400 uses the boundary for the representation.

In another embodiment, mapping module 400 may use auto-detected unique key points and descriptions of these key points to create a nearest neighborhood database in the map database 410. Each key point describes a particular feature of the environment near the autonomous vacuum 100 and the descriptions describe aspects of the features, such as color, material, location, etc. As the autonomous vacuum 100 moves about the environment, the mapping module 400 uses the visual data to extract unique key points and descriptions from the environment.

In some embodiments, the mapping module 400 may determine key points using a neural network. The mapping module 400 estimates which key points are visible in the nearest neighborhood database by using the descriptions as matching scores. After the mapping module 400 determines there are a threshold number of key points within visibility, the mapping module 400 uses these key points to determine a current location of the autonomous vacuum 100 by triangulating the locations of the key points with both the image location in the current visual data and the known location (if available) of the key point from the map database 415.

In another embodiment, the mapping module 400 uses the key points between a previous frame and a current frame in the visual data to estimate the current location of the autonomous vacuum 100 by using these matches as reference. This is typically done when the autonomous vacuum 100 is seeing a new scene for the first time, or when the autonomous vacuum 100 is unable to localize using previously existing key points on the map. Using this embodiment, the mapping module 400 can determine the position of the autonomous vacuum 100 within the environment at any given time. Further, the mapping module 400 may periodically purge duplicate key points and add new descriptions for key points to consolidate the data describing the key points. In some embodiments, this is done while the autonomous vacuum 100 is at the docking station 190.

The mapping module 400 processes the visual data when the autonomous vacuum 100 is at the docking station 190. The mapping module 400 runs an expansive algorithm to process the visual data to identify the objects and other features of the environment and piece them together into the map. The mapping module stores the map in the map database 415 and may store the map as a 3D satellite view of the environment. The mapping module 400 may update the map in the map database 415 to account for movement of objects in the environment upon receiving more visual data from the autonomous vacuum 100 as it moves around the environment over time. By completing this processing at the docking station, the autonomous vacuum 100 may save processing power relative to mapping and updating the map while moving around the environment. The mapping module 400 may use the map to quickly locate and/or determine the location of the autonomous vacuum 100 within the environment, which is faster than when computing the map at the same time. This allows the autonomous vacuum 100 to focus its processing power while moving on mess detection, localization, and user interactions while saving visual data for further analysis at the docking station 190.

The mapping module 400 constructs a layout of the environment as the basis of the map using visual data. The layout may include boundaries, such as walls, that define rooms, and the mapping module 400 layers objects into this layout to construct the map. In some embodiments, the mapping module 400 may use surface normals from 3D estimates of the environment and find dominant planes using one or more algorithms, such as RANSAC, which the mapping module 400 uses to construct the layout. In other embodiments, the mapping module 400 may predict masks corresponding to dominant planes in the environment using a neural network trained to locate the ground plane and surface planes on each side of the autonomous vacuum 100. If such surface planes are not present in the environment, the neural network may output an indication of “no planes.” The neural network may be a state-of-the-art object detection and masking network trained on a dataset of visual data labeled with walls and other dominant planes.

The mapping module 400 also uses the visual data to analyze surfaces throughout the environment. The mapping module 400 may insert visual data for each surface into the map to be used by the mess detection module 430 as it detects messes in the environment, described further below. For each different surface in the environment, the mapping module 400 determines a surface type of the surface and tags the surface with the surface type in the map. Surface types include various types of carpet, wood, tile, and cement, and, in some embodiments, the mapping module 400 determines a height for each surface type. For example, in a house, the floor of a dining room may be wood, the floor of a living room may be nylon carpet, and the floor of a bedroom may be a polyester carpet that is thicker than a nylon carpet in a hallway. The mapping module may also determine and tag surface types for objects in the room, such as carpets or rugs. The mapping module 400 sends the information about the surface types in the environment to the surface detection module 480.

The mapping module 400 further analyzes the visual data to determine the objects in the environment. Objects may include furniture, rugs, people, pets, and everyday household objects that the autonomous vacuum 100 may encounter on the ground, such as books, toys, and bags. Some objects may be barriers that define a room or obstacles that the autonomous vacuum 100 may need to remove, move, or go around, such as a pile of books. To identify the objects in the environment, the mapping module 400 predicts the plane of the ground in the environment using the visual data and removes the plane from the visual data to segment out an object in 3D.

In some embodiments, the mapping module 400 uses an object database to determine what an object is. In other embodiments, the mapping module 400 retrieves and compares visual data retrieved from an external server to the segmented objects to determine what the object is and tag the object with a descriptor. In further embodiments, the mapping module 400 may use the pretrained object detection module 405, which may be neural network based, to detect and pixel-wise segment objects such as chairs, tables, books, shoes. For example, the mapping module 400 may tag each of 4 chairs around a table as “chair” and the table as “table” and may include unique identifiers for each object (i.e., “chair A” and “chair B”).

In some embodiments, the mapping module 400 may also associate or tag an object with a barrier or warning. For example, the mapping module 400 may construct a virtual border around the top of a staircase in the map such that the autonomous vacuum 100 does not enter the virtual border to avoid falling down the stairs. As another example, the mapping module 400 may tag a baby with a warning that the baby is more fragile than other people in the environment.

The map may include three distinct levels for the objects in the environment: a long-term level, an intermediate level, and an immediate level. Each level may layer onto the layout of the environment to create the map of the entire environment. The long-term level contains a mapping of objects in the environment that are static. In some embodiments, an object may be considered static if the autonomous vacuum 100 has not detected that the object moved within the environment for a threshold amount of time (e.g., 10 days or more). In other embodiments, an object is static if the autonomous vacuum 100 never detects that the object moved. For example, in a bedroom, the bed may not move locations within the bedroom, so the bed would be part of the long-term level. The same may apply for a dresser, a nightstand, or an armoire. The long-term level also includes fixed components of the environment, such as walls, stairs, or the like.

The intermediate level contains a mapping of objects in the environment that are dynamic. These objects move regularly within the environment and may be objects that are usually moving, like a pet or child, or objects that move locations on a day-to-day basis, like chairs or bags. The mapping module 400 may assign objects to the intermediate level upon detecting that the objects move more often than a threshold amount of time. For example, the mapping module 400 may map chairs in a dining room to the intermediate level because the chairs move daily on average, but map the dining room table to the long-term level because the visual data has not shown that the dining room table has moved in more than 5 days. However, in some embodiments, the mapping module 400 does not use the intermediate level and only constructs the map using the long-term level and the immediate level.

The immediate level contains a mapping of objects within a threshold radius of the autonomous vacuum 100. The threshold radius may be set at a predetermined distance (i.e., 5 feet) or may be determined based on the objects the autonomous vacuum 100 can discern using the camera system 320 within a certain resolution given the amount of light in the environment. For example, the immediate level may contain objects in a wider vicinity around the autonomous vacuum 100 around noon, which is a bright time of day, than in the late evening, which may be darker if no indoor lights are on. In some embodiments, the immediate level includes any objects within a certain vicinity of the autonomous vacuum 100.

In other embodiments, the immediate level only includes objects within a certain vicinity that are moving, such as people or animals. For each person within the environment, the mapping module 400 may determine a fingerprint of the person to store in the fingerprint database 420. A fingerprint is a representation of a person and may include both audio and visual information, such as an image of the person's face (i.e., a face print), an outline of the person's body (i.e., a body print), a representation of the clothing the person is wearing, and a voice print describing aspects of the person's voice determined through voice print identification. The mapping module 400 may update a person's fingerprint in the fingerprint database 420 each time the autonomous vacuum 400 encounters the person to include more information describing the person's clothing, facial structure, voice, and any other identifying features.

In another embodiment, when the mapping module 400 detects a person in the environment, the mapping module 400 creates a temporary fingerprint using the representation of the clothing the person is currently wearing and uses the temporary fingerprint to track and follow a person in case this person interacts with the autonomous vacuum 100, for example, by telling the autonomous vacuum 100 to “follow me.” Embodiments using temporary fingerprints allow the autonomous vacuum 100 to track people in the environment even without visual data of their faces or other defining characteristics of their appearance.

The mapping module 400 updates the mapping of objects within these levels as it gathers more visual data about the environment over time. In some embodiments, the mapping module 400 only updates the long-term level and the intermediate level while the autonomous vacuum 100 is at the docking station, but updates immediate level as the autonomous vacuum 100 moves around the environment. For objects in the long-term level, the mapping module 400 may determine a probabilistic error value about the movement of the object indicating the chance that the object moved within the environment and store the probabilistic error value in the map database 415 in association with the object. For objects in the long-term map with a probabilistic error value over a threshold value, the mapping module 400 characterizes the object in the map and an area that the object has been located in the map as ambiguous.

In some embodiments, the object detection module 405 detects and segments various objects in the environment. Some examples of objects include tables, chairs, shoes, bags, cats, and dogs. In one embodiment, the object detection module 405 uses a pre-trained neural network to detect and segment objects. The neural network may be trained on a labeled set of data describing an environment and objects in the environment. The object detection module 405 also detects humans and any joint points on them, such as knees, hips, ankles, wrists, elbows, shoulders, and head. In one embodiment, the object detection module 405 determines these joint points via a pre-trained neural network system on a labeled dataset of humans with joint points.

In some embodiments, the mapping module 400 uses the optional spatial modeling module 410 to create a 3D rendering of the map. The spatial modeling module 410 uses visual data captured by stereo cameras on the autonomous vacuum 100 to create an estimated 3D rendering of a scene in the environment. In one embodiment, the spatial modeling module 410 uses a neural network with an input of two left and right stereo images and learns to produce estimated 3D renderings of videos using the neural network. This estimated 3D rendering can then be used to find 3D renderings of joint points on humans as computed by the object detection module 405. In one embodiment, the estimated 3D rendering can then be used to predict the ground plane for the mapping module 400. To predict the ground plane, the spatial modeling module 410 uses a known camera position of the stereo cameras (from the hardware and industrial design layout) to determine an expected height ground plane. The spatial modeling module 410 uses all image points with estimated 3D coordinates at the expected height as the ground plane. In another embodiment, the spatial modeling module 410 can use the estimated 3D rendering to estimate various other planes in the environment, such as walls. To estimate which image points are on a wall, the spatial modeling module 410 estimates clusters of image points that are vertical (or any expected angle for other planes) and groups connected image points into a plane.

In some embodiments, the mapping module 400 passes the 3D rendering through a scene-classification neural network to determine a hierarchical classification of the home. For example, a top layer of the classification decomposes the environment into different room types (e.g., kitchen, living room, storage, bathroom, etc.). A second layer decomposes each room according to objects (e.g., television, sofa, and vase in the living room and bed, dresser, and lamps in the bedroom). The autonomous vacuum 100 may subsequently use the hierarchical model in conjunction with the 3D rendering to understand the environment when presented with tasks in the environment (e.g., “clean by the lamp”). It is noted that the map ultimately may be provided for rendering on a device (e.g., wirelessly or wired connected) with an associated screen, for example, a smartphone, tablet, laptop or desktop computer. Further, the map may be transmitted to a cloud service before being provided for rendering on a device with an associated screen.

The mess detection module 430 detects messes within the environment, which are indicated by pixels in real-time visual data that do not match the surface type. As the autonomous vacuum 100 moves around the environment, the camera system 320 collects a set of visual data about the environment and sends it to the mess detection module 430. From the visual data, the mess detection module 430 determines the surface type for an area of the environment, either by referencing the map or by comparing the collected visual data to stored visual data from a surface database. In some embodiments, the mess detection module 430 may remove or disregard objects other than the surface in order to focus on the visual data of the ground that may indicate a mess. The mess detection module 430 analyzes the surface in the visual data pixel-by-pixel for pixels that do not match the pixels of the surface type of the area. For areas with pixels that do not match the surface type, the mess detection module 430 segments out the area from the visual data using a binary mask and compares the segmented visual data to the long-term level of the map. In some embodiments, since the lighting when the segmented visual data was taken may be different from the lighting of the visual data in the map, the mess detection module 430 may normalize the segmented visual data for the lighting. For areas within the segmented visual data where the pixels do not match the map, the mess detection module 430 flags the area as containing a mess and sends the segmented visual data, along with the location of the area on the map, to the task module 440, which is described below. In some embodiments, the mess detection module 430 uses a neural network for detecting dust in the segmented visual data.

For each detected mess, the mess detection module 430 verifies that the surface type for the area of the mess matches the tagged surface type in the map for that area. In some embodiments, if the surface types do not match to within a confidence threshold, the mess detection module 430 re labels the surface in the map with the newly detected surface type. In other embodiments, the mess detection module 430 requests that the autonomous vacuum 100 collect more visual data to determine the surface type to determine the surface type of the area.

The mess detection module 430 may also detect messes and requested cleaning tasks via user interactions from a user in the environment. As the autonomous vacuum 100 moves around the environment, the sensor system 118 captures ambient audio and visual data using the microphone 330 and the camera system 320 that is sent to the mess detection module 430. In one embodiment, where the microphone 330 is an array of microphones 330, the detection module 330 may process audio data from each of the microphones 330 in conjunction with one another to generate one or more beamformed audio channels, each associated with a direction (or, in some embodiments, range of directions). In some embodiments, the mess detection module 430 may perform image processing functions on the visual data by zooming, panning, de-warping.

When the autonomous vacuum 100 encounters a person in the environment, the mess detection module 430 may use face detection and face recognition on visual data collected by the camera system 320 to identify the person and update the person's fingerprint in the fingerprint database 440. The mess detection module 430 may use voice print identification on a user speech input a person (or user) to match the user speech input to a fingerprint and move to that user to receive further instructions. Further, the mess detection module 430 may parse the user speech input for a hotword that indicates the user is requesting an action and process the user speech input to connect words to meanings and determine a cleaning task. In some embodiments, the mess detection module 430 also performs gesture recognition on the visual data to determine the cleaning task. For example, a user may ask the autonomous vacuum 100 to “clean up that mess” and point to a mess within the environment. The mess detection module 430 detects and processes this interaction to determine that a cleaning task has been requested and determines a location of the mess based on the user's gesture. To detect the location of the mess, the mess detection module 430 obtains visual data describing the user's hands and eyes from the object detection module 405 and obtains an estimated 3D rendering of the user's hands and eyes from spatial modeling module 410 to create a virtual 3D ray. The mess detection module 430 intersects the virtual 3D ray with an estimate of the ground plane to determine the location the user is pointing to. The detection module 440 sends the cleaning task (and location of the mess) to the task module 440 to determine a mess type, surface type, actions to remove the mess, and cleaning settings, described below.

In some embodiments, the mess detection module 430 may apply a neural network to visual data of the environment to detect dirt in the environment. In particular, the mess detection module 430 may receive real-time visual data captured by the sensor system 118 (e.g., camera system and/or infrared system) and input the real-time visual data to the neural network. The neural network outputs a likelihood that the real-time visual data includes dirt, and may further output likelihoods that the real-time visual data includes dust and/or another mess type (e.g., a pile or spill) in some instances. For each of the outputs from the neural network, if the likelihood for any mess type is above a threshold, the mess detection module 430 flags the area as containing a mess (i.e., an area to be cleaned).

The mess detection module 430 may train the neural network on visual data of floors. In some embodiments, the mess detection module 430 may receive a first set of visual data from the sensor system 118 of an area in front of the autonomous vacuum 100 and a second set of visual data of the same area from behind the autonomous vacuum 100 after the autonomous vacuum 100 has cleaned the area. The autonomous vacuum 100 can capture the second set of visual data using cameras on the back of the autonomous vacuum or by turning around to capture the visual data using cameras on the front of the autonomous vacuum. The mess detection module 430 may label the first and second sets of visual data as “dirty” and “clean,” respectively, and train the neural network on the labeled sets of visual data. The mess detection module 430 may repeat this process for a variety of areas in the environment to train the neural network for the particular environment or for a variety of surface and mess types in the environment.

In another embodiment, the mess detection module 430 may receive visual data of the environment as the autonomous vacuum 100 clean the environment. The mess detection module 430 may pair the visual data to locations of the autonomous vacuum 100 determined by the mapping module 400 as the autonomous vacuum moved to clean. The mess detection module 430 estimates correspondence between the visual data to pair visual data of the same areas together based on the locations. The mess detection module 430 may compare the paired images in the RGB color space (or any suitable color or high-dimensional space that may be used to compute distance) to determine where the areas were clean or dirty and label the visual data as “clean” or “dirty” based on the comparison. Alternatively, the mess detection module 430 may compare the visual data to the map of the environment or to stored visual data for the surface type shown in the visual data. The mess detection module 430 may analyze the surface in the visual data pixel-by-pixel for pixels that do not match the pixels of the surface type of the area and label pixels that do not match as “dirty” and pixels that do match as “clean.” The mess detection module 430 trains the neural network on the labeled visual data to detect dirt in the environment.

In another embodiment, the mess detection module 430 may receive an estimate of the ground plane for a current location in the environment from the spatial modeling module 410. The mess detection module 430 predicts a texture of the floor of the environment based on the ground plane as the autonomous vacuum 100 moves around and labels visual data captured by the autonomous vacuum 100 with the floor texture predicted while the autonomous vacuum 100 moves around the environment. The mess detection module 430 trains the neural network on the labeled visual data to predict if a currently predicted floor texture maps to a previously predicted floor texture. The mess detection module 430 may then apply the neural network to real-time visual data and a currently predicted floor texture, and if the currently predicted floor texture does not map a previously predicted floor texture, the mess detection module 430 may determine that the area being traversed is dirty.

The task module 440 determines cleaning tasks for the autonomous vacuum 100 based on user interactions and detected messes in the environment. The task module 440 receives segmented visual data from the mess detection module 430 the location of the mess from the mess detection module 430. The task module 440 analyzes the segmented visual data to determine a mess type of the mess. Mess types describe the type and form of waste that comprises the mess and are used to determine what cleaning task the autonomous vacuum 100 should do to remove the mess. Examples of mess types include a stain, dust, a liquid spill, a solid spill, a smudge, or another type of debris. The mess types may include liquid waste, solid waste, or a combination of liquid and solid waste.

The task module 440 retrieves the surface type for the location of the mess from the map database and matches the mess type and surface type to a cleaning task architecture that describes the actions for the autonomous vacuum 100 to take to remove the mess. In some embodiments, the task module 440 uses a previous cleaning task from the task database for the given mess type and surface type. In other embodiments, the task module 440 matches the mess type and surfaces to actions the autonomous vacuum 100 can take to remove the mess and creates a corresponding cleaning task architecture of an ordered list of actions. In some embodiments, the task module 440 stores a set of constraints that it uses to determine cleaning settings for the cleaning task. The set of constraints describe what cleaning settings cannot be used for each mess type and surface type and how much force to apply to clean the mess based on the surface type. Cleaning settings include height of the cleaning head 140 and rotation speed of the brush roller 135 and the use of solvent 160. For example, the set of constraints may indicate that the solvent 160 can be used on wood and tile, but not on carpet, and the height of the cleaning head 140 must be at no more than 3 centimeters off the ground for cleaning stains in the carpet but at least 5 centimeters and no more than 7 centimeters off the ground to clean solid waste spills on the carpet.

Based on the determined actions and the cleaning settings for the mess, the task module 440 adds a cleaning task for each mess to task list database 450. The task list database 450 stores the cleaning tasks in a task list. The task list database 450 may associate each cleaning task with a mess type, a location in the environment, a surface type, a cleaning task architecture, and cleaning settings. For example, the first task on the task list in the task list database 450 may be a milk spill on tile in a kitchen, which the autonomous vacuum 100 may clean using solvent 160 and the brush roller 135. The cleaning tasks may be associated with a priority ranking that indicates how to order the cleaning tasks in the task list. In some embodiments, the priority ranking is set by a user via a client device 310 or is automatically determined by the autonomous vacuum 100 based on the size of the mess, the mess type, or the location of the mess. For example, the autonomous vacuum 100 may prioritize cleaning tasks in heavily trafficked areas of the environment (i.e., the living room of a house over the laundry room) or the user may rank messes with liquid waste with a higher priority ranking than messes with solid waste.

In some embodiments, the task module 440 adds cleaning tasks to the task list based on cleaning settings entered by the user. The cleaning settings may indicate which cleaning tasks the user prefers the autonomous vacuum 100 to complete on a regular basis or after a threshold amount of time has passed without a mess resulting in that cleaning task occurring. For example, the task module 440 may add a carpet deep cleaning task to the task list once a month and a hard wood polishing task to the task list if the hard wood has not been polished in more than some predetermined time period, e.g., 60 days.

The task module 440 may add additional tasks to the task list if the autonomous vacuum 100 completes all cleaning tasks on the task list. Additional tasks include charging at the docking station 190, processing visual data for the map via the mapping module 400 at the docking station 190, which may be done in conjunction with charging, and moving around the environment to gather more sensor data for detecting messes and mapping. The task module 440 may decide which additional task to add to the task list based on the amount of charge the battery 126 has or user preferences entered via a client device 310.

The task module 440 also determines when the autonomous vacuum 100 needs to be charged. If the task module 440 receives an indication from the battery 126 that the battery is low on power, the task module adds a charging task to the task list in the task list database 450. The charging task indicates that the autonomous vacuum 100 should navigate back to the docking station 190 and dock for charging. In some embodiments, the task module 440 may associate the charging task with a high priority ranking and move the charging task to the top of the task list. In other embodiments, the task module 440 may calculate how much power is required to complete each of the other cleaning tasks on the task list and allow the autonomous vacuum 100 to complete some of the cleaning tasks before returning to the docking station 190 to charge.

The navigation module 460 determines the location of the autonomous vacuum 100 in the environment. Using real-time sensor data from the sensor system 118, the navigation module 460 matches the visual data of the sensor data to the long-term level of the map to localize the autonomous vacuum 100. In some embodiments, the navigation module 460 uses a computer vision algorithm to match the visual data to the long-term level. The navigation module 460 sends information describing the location of the autonomous vacuum 100 to other modules within the controller 124. For example, the mess detection module 430 may use the location of the autonomous vacuum 100 to determine the location of a detected mess.

The navigation module 460 uses the immediate level of the map to determine how to navigate the environment to execute cleaning tasks on the task list. The immediate level describes the locations of objects within a certain vicinity of the autonomous vacuum 100, such as within the field of view of each camera in the camera system 320. These objects may pose as obstacles for the autonomous vacuum 100, which may move around the objects or move the objects out of its way. The navigation module interlays the immediate level of the map with the long-term level to determine viable directions of movement for the autonomous vacuum 100 based on where objects are not located. The navigation module 460 receives the first cleaning task in the task list database 450, which includes a location of the mess associated with the cleaning task. Based on the location determined from localization and the objects in the immediate level, the navigation module 100 determines a path to the location of the mess. In some embodiments, the navigation module 460 updates the path if objects in the environment move while the autonomous vacuum 100 is in transit to the mess. Further, the navigation module 460 may set the path to avoid fragile objects in the immediate level (e.g., a flower vase or expensive rug).

The logic module 470 determines instructions for the controller 124 to control the autonomous vacuum 100 based on the map in the map database 415, the task list database 450, and the path and location of the autonomous vacuum 100 determined by the navigation module 460. The instructions describe what each physical feature of the autonomous vacuum 100 should do to navigate an environment and execute tasks on the task list. Some of the physical features of the autonomous vacuum 100 include the brush motor 140, the side brush motor 150, the solvent pump 175, the actuator assembly 138, the vacuum pump 115, and the wheels 210. The logic module 470 also controls how and when the sensor system 118 collects sensor data in the environment. For example, logic module 470 may receive the task list from the task list database 450 and create instructions on how to navigate to handle the first cleaning task on the task list based on the path determined by the navigation module, such as rotating the wheels 210 or turning the autonomous vacuum 100. The logic module may update the instructions if the navigation module 460 updates the path as objects in the environment moved. Once the autonomous vacuum 100 has reached the mess associated with the cleaning task, the logic module 470 may generate instructions for executing the cleaning task. These instructions may dictate for the actuator assembly 138 to adjust the cleaning head height, the vacuum pump 115 to turn on, the brush roller 135 and/or side brush roller 154 to rotate at certain speeds, and the solvent motor 120 to dispense an amount of solvent 160, among other actions for cleaning. The logic module 470 may remove the cleaning task from the task list once the cleaning task has been completed and generate new instructions for the next cleaning task on the task list.

Further, the logic module 470 generates instructions for the controller 124 to execute. The instructions may include internal instructions, such as when to tick a clock node or gather sensor data, or external instructions, such as controlling the autonomous vacuum 100 to execute a cleaning task to remove a mess. The logic module 470 may retrieve data describing the map of the environment stored in the map database 415, fingerprint database 420, and task list database 450, or from other modules in the controller 124, to determine these instructions. The logic module 470 may also receive alerts/indications from other components of the autonomous vacuum 100 or from an external client device 310 that it uses to generate instructions for the controller 124. The logic module 470 may also send sensor data to one or more of the modules and send indications to the one or more modules when the autonomous vacuum is traversing in the environment, a real-time location of the autonomous vacuum 100, and the like.

The surface detection module 480 determines characteristics of surface types in an environment. Characteristics of a surface type may include, for example, material (e.g., marble, ceramic, oak, nylon, etc.), placement pattern (e.g., stacked squares, interlocking rectangles, etc.), grain/weave (e.g., density, visual texture and color variation), texture (shag, glossy, etc.), and color. The surface detection module 480 may determine the characteristics of the surface types of the environment in real-time or based on visual-inertial data saved in relation to the map of the environment.

In some embodiments, the surface detection module 480 receives real-time visual-inertial data of an environment from the logic module 470 as the autonomous vacuum 100 traverses the environment. The surface detection module 480 may also receive a real-time location of the autonomous vacuum in the environment from the logic module 470 and retrieve a surface type of the location based on the map stored in the map database 415. In other embodiments, the surface detection module 480 accesses visual-inertial data for a location of the environment corresponding to a surface type from the map database 415.

The surface detection module 480 analyzes the visual-inertial data to determine characteristics of the surface type. In some embodiments, the surface detection module 480 compares the visual data to images of a plurality of floors with the same surface type and selects characteristics of a floor with an image that is most similar to the visual data. In other embodiments, the surface detection module 480 inputs the visual data and surface type to a machine learning model configured to identify characteristics for a surface type. The machine learning model may output a likelihood for each characteristic of a plurality of characteristics that the floor depicted in the visual data includes the characteristic. The surface detection module 480 may compare the likelihoods to a threshold likelihood and select characteristics with a likelihood higher than the threshold likelihood for the surface type at the location. The surface detection module 480 may store the characteristics in association with the surface type and location in the map of the environment.

The machine learning model may be trained on image data labeled with characteristics of a surface type associated with the image data. For example, the machine learning model may be trained on a plurality of images of different floorings, each labeled with a surface type and characteristics. For instance, a first set of image data may be labeled with the surface type “tile” and the characteristics “porcelain,” “plank pattern,” “glossy,” and “black.” A second set of image data may be labeled with the surface type “tile” and the characteristics “porcelain,” “plank pattern,” “wood grain,” “wood texture,” and “natural white.” Though both floorings in these sets of image data are of the surface type “tile,” the floorings have some characteristics that differ. The surface detection module 480 may store and train the machine learning model on sample image data of floorings received from a plurality of external flooring systems or external builders and contractors via an API presented at a client device 310.

The surface detection module 480 may also analyze the inertial data to augment the characteristics of the surface types of the environment. For instance, surface detection module 480 may determine, based on the inertial data, that the autonomous vacuum 100 moves more quickly over the surface type “tile” than the surface type “carpet.” The surface detection module 480 may build an understanding of how the autonomous vacuum 100 moves on different surface types and compare its understanding to the captured inertial data. For instance, the surface detection module 480 may determine that the autonomous vacuum typically moves at a speed of 10 miles per hour on rugs when a specific torque (or power) is applied to motors of the wheels. As the autonomous vacuum 100 traverses the environment, the surface detection module 480 may compare a real-time speed of the autonomous vacuum 100 as the specific torque is applied to the motors to recorded speeds for locations of the environment. The surface detection module 480 determines whether the speed at a location varies by more than a threshold percentage from a recorded speed, and if so, the surface detection module 480 determines that the surface type may have changed. In such instances, the surface detection module 480 may redetermine the surface type at the location and update the map in the map database 415. In another example, the surface detection module 480 may compare the torque required to make the autonomous vacuum 100 go a certain speed (e.g., 2 miles per hour) to the specific torque stored in relation to that location and speed for the autonomous vacuum 100. If the torque varies by more than a threshold percentage, the surface detection module 480 may redetermine the surface type of the location.

The surface detection module 480 also may determine a height of each surface type in the environment, e.g., by analyzing surface type or measuring distance from a ground plane to top canopy of surface. The surface detection module 480 may transmit (or send) an indication to the logic module 470 to lower the height of the cleaning head 140 to be resting on a surface at a location in the environment. The surface detection module 480 may determine, based on sensor data of the from the sensor system 118 via the logic module 470, a height of the wheels of the autonomous vacuum 100 and a height of the cleaning head 140. For example, the surface detection module 480 may determine the height of the wheels based on IMU data and the height of the cleaning head 140 based on visual data. The surface detection module 480 may store the height of the wheel and cleaning head 140 in relation to the location in the map database 415. In some embodiments, the surface detection module 480 may use the height of the cleaning head 140 and the wheels to determine boundaries between surface types in the environment. For example, on a dense (or thick) carpet, the cleaning head 140 may be at a height above the carpet while the wheels may be at a height below the carpet (due to the autonomous vacuum 100 sinking into the carpet). The cleaning head 140 may move up in height as the cleaning head 140 is moved over a rug on top of the carpet, while the wheels may move up the same amount in height as the wheels move over the rug. The surface detection module 480 may detect such change in height of the wheels and cleaning head 140 and store information corresponding to the location of the height change in the map database 415 as indicating a boundary between surface types.

The surface detection module 480 may store cleaning information in the map database 415 in association with each surface type. The cleaning information may include a particular fan speed, a particular noise level, a list of which components of the autonomous vacuum 100 should be used to clean the surface type, and the like. The surface detection module 480 may retrieve the cleaning information from external flooring systems associated with the surface type or external builders and contractors who entered the cleaning information via the API on a client device 310. For example, cleaning instructions for the surface type “carpet” may indicate to use an internal fan of the autonomous vacuum and cleaning information for the surface type “wood” may indicate to operate components of the autonomous vacuum 100 at a lower volume than for the surface type “rug.” The surface detection module 480 may communicate the cleaning information to the logic module 470 such that the logic module may use the cleaning information to determine how to control components of the autonomous vacuum 100 for cleaning.

In some embodiments, the surface detection module 480 receives real-time visual data of the environment and determines how to clean an area of the environment based on the surface type. In particular, the surface detection module 480 receives mess types detected in the section from the mess detection module 430 and/or task module 440. Alternatively, the surface detection module 480 may input the visual-inertial data of the environment along with the surface type to a machine learning model trained to detect messes of different mess types. The machine learning model may be trained on visual data of a plurality of surface types each labeled with the surface type and which pixels correspond to messes and of what mess types.

The machine learning model outputs a likelihood that the visual data includes a mess and likelihoods representing which mess type is shown. The surface detection module 480 compares the likelihood of including mess to a mess threshold, and if the threshold is exceeded, compares the likelihoods representing the mess types to a mess type threshold. The surface detection module 480 selects mess types associated with likelihoods over the mess type threshold. The surface detection module 480 accesses cleaning information for the determined surface type and mess type and sends the cleaning information to the logic module 470 to control the autonomous vacuum 100 to execute techniques based on the cleaning information. For instance, for a mess type of “spilled crumbs,” the cleaning information may indicate to vacuum over the mess and subsequently mop over the mess, which the flooring module may instruct the logic module 470 to control the autonomous vacuum 100 to do.

The surface detection module 480 may monitor the characteristics of the surface types in the environment as the autonomous vacuum 100 traverses the environment. For instance, the surface detection module 480 may use the machine learning model on image data captured in real-time of the environment and compare the characteristics determined by the machine-learning model to the characteristics stored from a corresponding location in the map database 415. If the surface detection module 480 detects a discrepancy in characteristics (e.g., new or missing characteristics), the surface detection module 480 may determine whether the autonomous vacuum 100 is traversing a boundary between surface types I the environment based on the height of the cleaning head and wheels and/or the visual data. The surface detection module 480 may redetermine the surface type for the location and update the map database 415 to indicate the surface type. The surface detection module 480 may send an indication of the boundary and/or redetermined surface type of the user interface module 490.

The user interface module 490 generates user interfaces for the client devices 320 and display 122. The user interfaces (or APIs) may include renderings of the environment, including messes, obstacles, and the like, retrieved from the map database 415. A rendering may be shown as a top-down view of the environment including representations of each surface type in corresponding areas of the environment. The user interfaces may also include a plurality of interactive elements that a user may interact with to control movement of the autonomous vacuum 100 and view sensor data captured by the autonomous vacuum 100. Examples of user interfaces are shown in FIGS. 7-8B. The user interface module 490 generated and transmits user interfaces in response to receiving indications from the client devices 320 and/or display 122 and updates the user interfaces based on interactions received from the client devices 320 and/or display 122.

The user interface module 490 may generate user interfaces that depict a surface type in a background. For instance, the user interface module 490 may receive a real-time location of the autonomous vacuum 100 from sensor system 118 via the logic module 470. The user interface module 490 may retrieve, from the map database 415, a surface type and characteristics of the location. The user interface module 490 may also receive real-time visual data off the surface type at or around the location from the sensor system 118 via the logic module 470. The user interface module 490 may insert a portion of image data corresponding to the surface type to a user interface as the background of the user interface. In some embodiments, the user interface module 490 may align the portion of image data to match an orientation of the pattern of the surface type in the real-time visual data. For instance, the background of the user interface may depict the surface type with a pattern in the same orientation as seen by a camera at the front 200 of the autonomous vacuum 100.

The user interface module 490 may layer interactive elements (or icons) on top of the background. In some embodiments, the interactive elements may remain stationary over the background of the user interface as the background renders a scroll animation, as described above. The interactive elements receive interactions from a user via the user interface that indicate to instruct the autonomous vacuum 100 and/or request data about the autonomous vacuum's 100 cleaning in the environment. In some embodiments, the user interface module 490 may alternately or additionally insert an element indicating the surface type to the user interface.

In some embodiments, the user interface module 490 may configure the image of the surface type in the background to slide within the background based on movement of the autonomous vacuum 100. For instance, the user interface module 490 may determine a real-time speed of the autonomous vacuum from IMU, motor, and/or visual data captured by the sensor system 118 and sent to the user interface module 490 via the logic module 470. The user interface module 490 may configure the image data of the background to be animated at a speed that is linearly related to the real-time speed. The user interface module 490 may further configure the image data in the background to mirror an alignment of flooring in the environment based on real-time image data of the environment. For example, the user interface module may align grout lines from the image data in the background with grout lines in the real-time image data, such that the patterns shown in the background and the real-time image data match. In another example, the user interface module 490 may determine an orientation of fibers in a carpet and align image data of the carpet in the background of the user interface to match real-time image data of the carpet captured at the front 200 of the autonomous vacuum 100.

In some embodiments, the user interface module 490 includes one or more interactive elements for users, such as the user of the autonomous vacuum 100 or external builders or operators, to enter characteristics and cleaning information for surface types in an environment. For instance, the user interface module 490 may generate a user interface with a rendering of the environment with the floor in the rendering divided into selectable sections. Each selectable section may correspond to a surface type determined for an area of the environment. The user interface module 490 may receive a selection of a selectable section and insert information about the surface type and characteristics of the area with the surface type in the user interface. The user interface module 490 may also add one or more text boxes or selectable lists to the user interface that a use may interact with to add more characteristics and cleaning information to the map database 415 for the surface type and section. For instance, a contractor may change a surface type and characteristics of the section after replacing flooring in the environment.

In some embodiments, the user interface module 490 may communicate with a camera of the client device 310 presenting the user interface to receive machine-readable codes. The user interface module 490 may communicate the machine-readable codes to external systems (e.g., the Internet) to collet information associated with the machine-readable codes (e.g., characteristics and cleaning information. For example, a contractor, after renovating the environment by replacing carpet with tile, selects an area of the environment in a rendering presented the user interface. The user interface module 490 receives the selection and edits the user interface to include a scan icon that communicates with a camera of a client device 310 presenting the user interface. The contractor may scan, using the scan icon, a machine-readable code associated with the tile. The user interface module 490 receives the machine-readable code and retrieves information about the tile from a website associated with the machine-readable code. The user interface module 490 updates the map of the environment to include the surface type (e.g., “tile”) and characteristics and cleaning information from the retrieved information.

In some embodiments, the user interface module 490 may generate a user interface including a joystick element that is configured to receive interactions corresponding to desired movements of the autonomous vacuum 100. For example, the user interface module 490 may receive an interaction from the joystick element indicating movement to the left and communicate with the logic module 470 to move the autonomous vacuum 100 to the left tin the environment. The user interface module 490 may compare movements corresponding to interactions with the joystick button to the map of the environment based on a real-time location of the autonomous vacuum 100. If a movement would lead to an error for the autonomous vacuum 100 (e.g., surpassing a virtual border, hitting a wall, running the mop roller on carpet, etc.), the user interface module 490 may send an alert of the error via the user interface rather than proceeding to instruct the logic module 470 based on the movement.

The user interface may also include one or more interactive elements for manually controlling other components of the autonomous vacuum, such as the cleaning rollers, solvent motor 120, vacuum pump 115, actuator assembly 138, and sensors of the sensor system 118. For example, the user interface module 490 may receive an interaction with a mop roller button via the user interface and send an indication to the logic module 470 to activate a mop roller of the autonomous vacuum 100. The user interface may also include interactive elements for controlling a noise level emitted by the autonomous vacuum 100 during cleaning. For instance, the user interface module 490 may include an interactive slider configured to raise or lower the noise emitted from the autonomous vacuum 100 during operation as the interactive slider is slid to one direction or the other, respectively. The user interface module 490 may communicate with the logic module 470 to control the noise of components of the autonomous vacuum 100 and may send notifications via the user interface regarding change sin noise level.

### EXAMPLE MUFFLER

In one example embodiment a muffler is coupled to backend of the vacuum motor 120 to dampen noise prior to exhausting the air into an external environment of the autonomous vacuum 100. The muffler may include one or more chambers to fluidically move the air from the vacuum motor 120 to the exhaust outlet and one or more dampening elements to absorb sound waves from the air.

FIG. 5 is a cross-sectional view of an airflow pathway 500 for the autonomous vacuum, according to one or more embodiments. The airflow pathway 500 is illustrated with the grey arrows flowing into the cleaning head 140 (e.g., via the sweeper roller disposed towards a front of the cleaning head 140 and/or the mop roller disposed towards a back of the cleaning head 140), through the channels 505 (e.g., the dry channel 132 and/or the liquid channel(s) 134 of the connection assembly 130), into the waste bag 112 situated in a bin cavity 520 via a bin inlet 522, through a vacuum stack inlet 524 to the vacuum motor 120, through a muffler 540 that dampens sound waves in the air, and out of the autonomous vacuum through an exhaust 550.

The waste bag 112 is connected in the airflow pathway 500. Being situated in the bin cavity 520, the waste bag 112 can operate as a filter allowing debris to be collected into the waste bag 112 whilst filtered air passes through an air-permeable membrane of the waste bag 112. The pores of the permeable membrane determine the filter efficiency (typically rated in HEPA) and airflow characteristics. The smaller the pore size, the higher the filter efficiency and lower the airflow for the same surface area.

FIG. 6A is an exploded view of a muffler 600 for dampening noise created by the vacuum motor, according to one or more embodiments. FIG. 6B is a constructed view of the muffler 600, according to one or more embodiments. The muffler 600 is one embodiment of the muffler 540 of FIG. 5. As shown in the exploded view of FIG. 6A, the muffler 600 includes a main body 610. A first side of the main body 610 includes an inlet 605 and a second side of the main body includes a cavity. The cavity may be divided into a plurality of fluidic chambers. Within the cavity is a rigid layer 615 separating two or more of the chambers. On top of the rigid layer 615 is a fabric layer 620. The fabric layer 620 may be directly on top of the rigid layer 615 or there may be a gap, e.g., an air gap, between the two layers 615, 620. The fabric layer 620 fits within the cavity of the main body 610. The rigid layer 615 includes an array of holes for fluidic communication between the two or more chambers The fabric layer 620 includes an array of perforations.

On top of the fabric layer 620 is a foam layer 630 that also fits within the cavity of the main body 610. The foam layer 630 includes a cutaway 635 that is on a first end along a first side. A sealing plate 640 covers the cavity of the main body 610. The sealing plate 640 includes an outlet 645. The outlet 645 is an opening from the sealing plate 640. A foam 650 may fit along a length of the outlet 645 to cover the outlet.

In other embodiments, the muffler 600 may include additional, fewer, or different components. For example, the muffler 600 may omit either the fabric layer 620, the rigid layer 615, the foam layer 630, or the foam 650. In other examples, the muffler 600 may include additional dampening elements beyond the fabric layer 620, the foam layer 630, and the foam 650.

The main body 610 and the sealing plate 640 provide the overarching structure to the muffler 600. The main body 610 and/or the sealing plate 640 may be formed of rigid materials, e.g., metal, plastic, glass, etc. The main body 610 includes an inlet 605 for receiving air from the vacuum motor, whereas the sealing plate 640 includes an outlet 645 for exhausting air into an external environment. The main body 610 and the sealing plate 640 may form one or more fluidic chambers within the cavity and disposed in the airflow pathway between the inlet 605 and the outlet 645. For example, the main body 610 may include a bottom chamber and an middle chamber (further described in FIGS. 7A and 7B). Each chamber may include dampening elements to absorb sound waves from the air exhausted by the vacuum motor. The inlet 605 couples to the vacuum stack inclusive of the vacuum motor. As shown in FIG. 6A, the inlet 605 may have a cross-sectional area that matches to the vacuum stack. The inlet 605 is fluidically connected to the bottom chamber. The bottom chamber and the middle chamber are separated by a rigid layer 615. The bottom chamber and/or the middle chamber may be substantially shallow, i.e., a height of the chamber is substantially smaller than the length and the width. The rigid layer 615 may include an array of holes that fluidically connect the bottom chamber to the middle chamber.

The fabric layer 620 is formed of a fabric material that dampens sound waves from air that contacts the fabric layer 620. Example fabric materials include: cotton, felt, wool, mass-loaded vinyl, acoustical cotton batts, fiberglass, polyester fiber, etc. In one or more embodiments, the fabric layer 620 is positioned atop the rigid layer 615, i.e., within the middle chamber. In other embodiments, the fabric layer 620 may be positioned in the bottom chamber. In other embodiments, there can be an additional fabric layer—one for coupling to the bottom chamber and one for coupling to the middle chamber. The fabric layer 620 may include an array of perforations 625 for increasing airflow through the fabric layer 620. In some embodiments, the array of perforations 625 aligns with holes of the rigid layer 615. In the embodiment shown in FIG. 6A, the fabric layer 620 may include a cutaway portion that fits around a protrusion in the main body 610 for placement of a caster wheel in the chassis of the autonomous vacuum.

The foam layer 630 if formed of a foam material that dampens sound waves from air that contacts the foam layer 630. Example foam materials include: open-cell foam, closed-cell foam, polyurethane foam, melamine foam, and acoustic foam. The foam layer 630 may be coupled to a bottom surface of the sealing plate 640, e.g., with an adhesive. The foam layer 630 may include a cutaway portion 635 for fluidic connection of the middle chamber with the outlet 645 of the sealing plate 640. The cutaway portion 635 may be sized to match a cross-sectional area of the outlet 645. As shown in FIGS. 6A & 6B, the outlet 645 is positioned on the sealing plate 640 towards a middle of the muffler 600 along the x-axis.

The sealing plate 640 includes the outlet 645 to exhaust the noise-dampened air to the external environment. The sealing plate 640 includes a plurality of attachment points, e.g., screw holes for screwing down the sealing plate 640 to the main body 610, or ultrasonic welding of sealing plate 640 to the main body 610. The outlet 645 is formed by a hollow protrusion extending from a planar surface. The outlet 645 may be angled to exhaust the air at an angle upward, to avoid the exhaust spreading debris on the ground surface. The sealing plate 640 and main body 610 may also be tuned in terms of stiffness to avoid resonance caused by vibrations from the vacuum motor 120.

While moving in a grid like pattern, also known as “coverage path” on a floor, the autonomous vacuum goes over all of the surface with its cleaning apparatus. As the autonomous vacuum traverses along the edge separating portions of the floor already cleaned and portions yet to be cleaned, the airflow exhausting from the autonomous vacuum can possibly displace the dirt from the to-be-cleaned area into the already-cleaned area.

This creates a number of problems. First, the dirt displaced may not get cleaned until the next cleaning operation, as the autonomous vacuum would not be re-clean a previously-cleaned area. Second, dirt blowing up might lead the dirt being deposited on surface(s) that are completely unreachable by the autonomous vacuum, e.g., atop furniture, counters, or tables. Third, dirt blown up may be suspended in the air, adding contaminants to the air and, consequently, reducing air quality for the inhabitants of the space.

In order to solve these problems, the outlet 645 angles the exhaust air upwards, e.g., at a slight angle. With air exhausted directly vertical, an inhabitant may have a bad experience with the exhausted air blown into their face. With air exhausted directly horizontal, the exhausted air runs into the potential pitfall of displacing dirt. Accordingly, the angled trajectory provides balance between the two pitfalls.

FIG. 7A is a cross-sectional view of the muffler 600 along a first dimension, according to one or more embodiments. FIG. 7B is a cross-sectional view of the muffler 600 along a second dimension perpendicular to the first dimension, according to one or more embodiments. The first dimension is a bisection by the xz-plane shown in the coordinate grid in FIG. 6B, i.e., showing a lengthwise cross-section. The second dimension is a bisection by the yz-plane shown in the coordinate grid in FIG. 6B, i.e., showing a widthwise cross-section.

FIGS. 7A & 7B illustrate the bottom chamber 710 and the middle chamber 720 formed in the muffler 600. As described above, the bottom chamber 710 may be formed into the main body 610. The rigid layer 615 separates the bottom chamber 710 from the middle chamber 720. The rigid layer 615 includes the array of holes that fluidically connects the bottom chamber 710 to the middle chamber 720.

During operation, the vacuum motor pushes air into the inlet (e.g., on a left-side of the cross-section view shown in FIG. 7A). The air travels into the bottom chamber 710. Then the air is passed through the holes in the rigid layer 615 and the array of perforations 625 of the fabric layer 620. The perforations 625 are designed to create an even airflow and to guide air path so that it hits the bottom surface of the foam layer 730 at high velocity. This interaction of sound waves with the foam aids in noise absorption.

When sound waves hit the bottom surface of the foam layer 730, part of the sound waves may get absorbed, part of the sound waves may get transmitted, and part of the sound waves may be reflected. The aim is to increase the absorptivity as much as possible and to avoid any sound waves transmitting through the foam layer 730. The foam material may be selected to be one of high absorptivity coefficient.

The portion of sound waves that is reflected from the foam layer 630 collides with the fabric layer 620. This further reduces the noise with multiples collisions with noise-absorbing elements. The more the sound waves bounce back and forth, the more energy is absorbed into the foam layer 630 and/or the fabric layer 620.

After sound absorption, the air is pushed towards the outlet 645. The outlet 645 may include an additional foam 650 placed on a top portion of the outlet 645. The foam can further aid in noise absorption prior to the air exhausting to the external environment.

### EXAMPLE CLAUSES

Clause 1. An autonomous vacuum comprising: a vacuum motor; and a muffler coupled to the vacuum motor for exhausting air and for noise reduction, the muffler comprising: a main body including an inlet for air exhausted by the vacuum motor and forming one or more chambers fluidically connected to the inlet; a foam layer for noise absorption and disposed within one of the chambers of the main body; and a sealing plate affixed to the main body including an outlet to exhaust air from the chambers to an external environment of the autonomous vacuum.

Clause 2. The autonomous vacuum of clause 1, wherein the main body comprises a rigid layer with an array of holes separating two chambers: a bottom chamber and a middle chamber, wherein the bottom chamber is fluidically connected to the inlet and the middle chamber is fluidically connected to the outlet.

Clause 3. The autonomous vacuum of clause 2, wherein the foam layer is disposed within the middle chamber and coupled to a bottom surface of the sealing plate.

Clause 4. The autonomous vacuum of clause 2, the muffler further comprising: a fabric layer coupled to the rigid layer for additional noise absorption.

Clause 5. The autonomous vacuum of clause 4, wherein the fabric layer is disposed within the middle chamber and coupled to a top surface of the rigid layer.

Clause 6. The autonomous vacuum of clause 4, wherein the fabric layer includes an array of perforations aligned to the array of holes of the rigid layer.

Clause 7. The autonomous vacuum of clause 4, wherein the fabric layer is formed of a fabric material selected from at least one of: cotton, felt, wool, mass-loaded vinyl, acoustical cotton batts, fiberglass, or polyester fiber.

Clause 8. The autonomous vacuum of clause 1, wherein the foam layer is formed of a foam material selected from at least one of: open-cell foam, closed-cell foam, polyurethane foam, melamine foam, or acoustic foam.

Clause 9. The autonomous vacuum of clause 1, wherein each chamber has a substantially small height relative to a length and a width of the chamber.

Clause 10. The autonomous vacuum of clause 1, the muffler further comprising: foam disposed within the outlet for added noise absorption.

Clause 11. A muffler coupled to a vacuum motor of an autonomous vacuum for exhausting air and for noise reduction, the muffler comprising: a main body including an inlet for air exhausted by the vacuum motor and forming one or more chambers fluidically connected to the inlet; a foam layer for noise absorption and disposed within one of the chambers of the main body; and a sealing plate affixed to the main body including an outlet to exhaust air from the chambers to an external environment of the autonomous vacuum.

Clause 12. The muffler of clause 11, wherein the main body comprises a rigid layer with an array of holes separating two chambers: a bottom chamber and a middle chamber, wherein the bottom chamber is fluidically connected to the inlet and the middle chamber is fluidically connected to the outlet.

Clause 13. The muffler of clause 12, wherein the foam layer is disposed within the middle chamber and coupled to a bottom surface of the sealing plate.

Clause 14. The muffler of clause 12, further comprising: a fabric layer coupled to the rigid layer for additional noise absorption.

Clause 15. The muffler of clause 14, wherein the fabric layer is disposed within the middle chamber and coupled to a top surface of the rigid layer.

Clause 16. The muffler of clause 14, wherein the fabric layer includes an array of perforations aligned to the array of holes of the rigid layer.

Clause 17. The muffler of clause 14, wherein the fabric layer is formed of a fabric material selected from: cotton, felt, wool, mass-loaded vinyl, acoustical cotton batts, fiberglass, or polyester fiber.

Clause 18. The muffler of clause 11, wherein the foam layer is formed of a foam material selected from: open-cell foam, closed-cell foam, polyurethane foam, melamine foam, or acoustic foam.

Clause 19. The muffler of clause 11, wherein each chamber has a substantially small height relative to a length and a width of the chamber.

Clause 20. The muffler of clause 11, further comprising: foam disposed within the outlet for added noise absorption.

### ADDITIONAL CONSIDERATIONS

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. An autonomous vacuum comprising:
a vacuum motor; and
a muffler coupled to the vacuum motor for exhausting air and for noise reduction, the muffler comprising:
a main body including an inlet for air exhausted by the vacuum motor and forming one or more chambers fluidically connected to the inlet;
a foam layer for noise absorption and disposed within one of the chambers of the main body; and
a sealing plate affixed to the main body including an outlet to exhaust air from the chambers to an external environment of the autonomous vacuum.

2. The autonomous vacuum of claim 1, wherein the main body comprises a rigid layer with an array of holes separating two chambers: a bottom chamber and a middle chamber, wherein the bottom chamber is fluidically connected to the inlet and the middle chamber is fluidically connected to the outlet.

3. The autonomous vacuum of claim 2, wherein the foam layer is disposed within the middle chamber and coupled to a bottom surface of the sealing plate.

4. The autonomous vacuum of claim 2, the muffler further comprising:
a fabric layer coupled to the rigid layer for additional noise absorption.

5. The autonomous vacuum of claim 4, wherein the fabric layer is disposed within the middle chamber and coupled to a top surface of the rigid layer.

6. The autonomous vacuum of claim 4, wherein the fabric layer includes an array of perforations aligned to the array of holes of the rigid layer.

7. The autonomous vacuum of claim 4, wherein the fabric layer is formed of a fabric material selected from at least one of: cotton, felt, wool, mass-loaded vinyl, acoustical cotton batts, fiberglass, or polyester fiber.

8. The autonomous vacuum of claim 1, wherein the foam layer is formed of a foam material selected from at least one of: open-cell foam, closed-cell foam, polyurethane foam, melamine foam, or acoustic foam.

9. The autonomous vacuum of claim 1, wherein each chamber has a substantially small height relative to a length and a width of the chamber.

10. The autonomous vacuum of claim 1, the muffler further comprising:
foam disposed within the outlet for added noise absorption.

11. A muffler coupled to a vacuum motor of an autonomous vacuum for exhausting air and for noise reduction, the muffler comprising:
a main body including an inlet for air exhausted by the vacuum motor and forming one or more chambers fluidically connected to the inlet;
a foam layer for noise absorption and disposed within one of the chambers of the main body; and
a sealing plate affixed to the main body including an outlet to exhaust air from the chambers to an external environment of the autonomous vacuum.

12. The muffler of claim 11, wherein the main body comprises a rigid layer with an array of holes separating two chambers: a bottom chamber and a middle chamber, wherein the bottom chamber is fluidically connected to the inlet and the middle chamber is fluidically connected to the outlet.

13. The muffler of claim 12, wherein the foam layer is disposed within the middle chamber and coupled to a bottom surface of the sealing plate.

14. The muffler of claim 12, further comprising:
a fabric layer coupled to the rigid layer for additional noise absorption.

15. The muffler of claim 14, wherein the fabric layer is disposed within the middle chamber and coupled to a top surface of the rigid layer.

16. The muffler of claim 14, wherein the fabric layer includes an array of perforations aligned to the array of holes of the rigid layer.

17. The muffler of claim 14, wherein the fabric layer is formed of a fabric material selected from: cotton, felt, wool, mass-loaded vinyl, acoustical cotton batts, fiberglass, or polyester fiber.

18. The muffler of claim 11, wherein the foam layer is formed of a foam material selected from: open-cell foam, closed-cell foam, polyurethane foam, melamine foam, or acoustic foam.

19. The muffler of claim 11, wherein each chamber has a substantially small height relative to a length and a width of the chamber.

20. The muffler of claim 11, further comprising:
foam disposed within the outlet for added noise absorption.
